(19) United States
(12) Patent Application Publication
(10) Pub. No.: US 2026/0216398 A1
(43) Pub. Date: Jul. 30, 2026

(54) Fabrication and Processing of Magnesium-Based Metal Matrix Nanocomposites for Bioabsorbable Implants

(71) Applicant: UNIVERSITY OF CENTRAL FLORIDA RESEARCH FOUNDATION, INC., Orlando, FL (US)

(72) Inventors: Mehdi RAZAVI, Orlando, FL (US); Andres LARRAZA, Orlando, FL (US); Pedram SOTOUDEHBAGHA, Orlando, FL (US)

(21) Appl. No.: 19/038,209

(22) Filed: Jan. 27, 2025

(51) Int. Cl. A61L 27/04; A61F 2/28; C01B 33/24; C22C 23/06

(52) U.S. Cl. CPC A61L 27/047; A61F 2/28; C01B 33/24; C22C 23/06

(57) ABSTRACT

Disclosed is a novel magnesium (Mg)-based metal-matrix nanocomposite (MMNC) fabricated using ultrasound melt treatment to promote the de-agglomeration of the bioactive glass-ceramic nanoparticles and the homogenization of the melt. The cast samples were then heat treated, machined, and hot rolled to reduce grain size and remove structural defects. Standard mechanical and electrochemical tests were conducted to determine the effect of fabrication and processing on mechanical and corrosion properties of MMNCs. Compression tests were conducted to measure yield strength, and ultimate compressive strength, and elongation at break. Potentiodynamic polarization tests, electrochemical impedance spectroscopy, and static immersion testing were conducted to determine the corrosion rate. Results showed that the combination of ultrasonic melt processing and thermomechanical processing significantly affected the corrosion behavior and mechanical properties of samples. Samples that underwent both processes had significantly higher yield stress and ultimate compressive strength and had significant changes in the corrosion rate at each time point measured. Implants comprising the MMNC and uses thereof are also disclosed.

### STATEMENT OF GOVERNMENT SUPPORT

The invention was made with government support under Grant No. CMMI 2142610 awarded by the National Science Foundation. The government has certain rights in the invention.

### BACKGROUND

Metal implants are predominantly used for bone-fixing devices due to their high mechanical properties [1,2]. Common alloys consist mainly of bioinert permanent materials like titanium alloys (Ti-6Al-4V) and stainless steels. These metals provide mechanical rigidity and chemical stability, which the bone needs to heal properly [3,4,5,6]. However, since these metals do not degrade over time, revisionary surgeries to remove the implants when the wound is fully healed are often necessary. Similarly, the release of metallic particles and ions from the surfaces poses a risk of adverse effects [7,8]. To combat these risks, developing a biocompatible, biodegradable metal can eliminate the need for revisionary surgery and overcome limitations associated with permanent implants [9].

Magnesium has shown significant potential in biodegradable implant applications due to its high biocompatibility; however, its potential is hindered by rapid corrosion rates and the generation of hydrogen gas as a corrosion byproduct [2,7]. Efforts to improve corrosion resistance through alloying and surface coating have yielded significant advances, but these enhancements often compromise biocompatibility [10,11,12]. WE43 is a commercially available biocompatible magnesium alloy that utilizes rare earth metals to reduce corrosion, rendering it nearly bioinert. Recent research is focused on fine-tuning corrosion resistance while maintaining biodegradability and enhancing osteogenic and angiogenic properties [13]. The addition of scandium (Sc) and strontium (Sr) has been shown to provide corrosion resistance in vitro while also providing biocompatible environments for osteoblast differentiation and offering antibacterial properties [14,15].

Current methods for fabricating magnesium alloys or composites typically involve either stir casting or powder metallurgy. In stir casting, an impeller is submerged into the molten material and rotated at high speeds to promote thorough mixing. Alternatively, powder metallurgy involves milling raw materials into fine powders, compacting them, and then sintering to achieve the desired density and porosity. A major drawback of stir casting is the introduction of gas bubbles into the melt, which leads to porosity during solidification [16,17,18]. This results in reduced mechanical strength and corrosion resistance, issues that are challenging to address within the casting process itself. In powder metallurgy, porosity is reduced by sintering the compacted sample, typically under high temperatures and pressures. This process increases sample density; however, achieving zero porosity remains challenging [19].

The application of melt processing techniques, such as ultrasound treatment, enhances the de-agglomeration and dispersion of additive particles, influences the grain morphology of as-cast materials, and eliminates structural defects like porosity and non-uniformities [19,20,21]. The stimulation of the melt with acoustic waves induces acoustic streaming, a phenomenon where the fluid solution moves in a regular oscillatory motion, which can result in cavitation of gas bubbles and de-agglomeration of reinforcing nanoparticles in the melt [22,23]. Post-melt processing via hot rolling also significantly affects the grain structure, which in turn affects the mechanical properties and corrosion behavior of the metals [24,25].

Although the individual alloying of magnesium with strontium (Sr) and scandium (Sc) has been studied for improvements in corrosion resistance, mechanical properties, and biocompatibility [26,27,28,29], the synergistic potential of these elements in a ternary Mg—Sr—Sc alloy system remains largely unexplored. Moreover, to our knowledge, no substantial studies have investigated the integration of this alloy with bioactive glass-ceramic nanoparticles. By developing this novel composite, we aim to harness the complementary benefits of Sr and Sc alloying together with bioactive glass-ceramic reinforcement, creating a multifunctional implant material that meets both the structural and biological demands of bone regeneration.

Incorporating diopside (CaMgSi₂O₆) bioactive glass-ceramic nanoparticles (BG) into a magnesium-based metal matrix nanocomposite (MMNC) offers significant potential benefits for implant biocompatibility and bioactivity. The sustained release of calcium, magnesium, and silicon ions from these nanoparticles can enhance osteogenesis and angiogenesis and improve biocompatibility [30,31,32].

### BRIEF DESCRIPTION OF DRAWINGS

FIG. 1. Microstructures of WE43 (A,B), Mg (C,D), unprocessed MMNC (E,F), UST MMNC (G,H), and UST Rolled MMNC (1,J) at 200× (A,C,E,G,I) and 700× (B,D,F,H,J) magnification. The images show grain sizes, intermetallics, and nano-reinforcements, with white arrows indicating nano-reinforcements and red arrows intermetallic formation at grain boundaries. The inset in panel J shows a higher magnification image (1500×). (K) Grain size measurements conducted according to ASTM E112 standards, calculated as mean lineal intercepts from 11 randomly selected replicates obtained from the metallographic images. **p≤0.01, ****p≤0.0001.

FIG. 2. SEM images and chemical point analysis of unprocessed MMNC (A-C), UST MMNC (D-F), and UST Rolled MMNC (G-1). The white insets in (A,D,G) are enlarged in (B,E,H), respectively. The red insets in (B,E,H) indicate the analyzed points using EDS represented in (C), (F), and (1), respectively. The maximum intensity of the EDS spectra was decreased to 200 counts for a better illustration of trace elements. TEM image of BG nanoparticles after synthesis and calcination at 850° C. for 2 h (J).

FIG. 3. (A) Stress-strain curves for WE43, Mg, unprocessed MMNC, UST MMNC, and UST Rolled MMNC, along with their corresponding calculated values for yield stress (B), ultimate compressive strength (UCS) (C), and % elongation at break (D). Nanocomposite fabrication significantly enhanced UCS, with minimal impact on yield stress. Ultrasonication of the MMNC melt improved mechanical properties compared to Mg but showed no significant difference compared to unprocessed MMNCs. The introduction of hot rolling increased yield stress and reduced % elongation, while applying minimal influence on UCS. One-way ANOVA: *p≤0.05, **p≤0.01, ****p≤0.0001; n=5 per group.

FIG. 4. (A) PDP curves for WE43, Mg, unprocessed MMNC, UST MMNC, and UST Rolled MMNC, illustrating the average corrosion behavior of samples during electrochemical testing. (B) Corrosion rate (in mm per year, mmpy), showing a significant reduction for UST Rolled MMNCs compared to other MMNCs. (C) Corrosion current density (Icorr) follows similar trends, with a significant decrease for UST Rolled MMNCs. (D) Corrosion potential (Ecorr) is elevated in UST Rolled MMNCs relative to the other samples. One-way ANOVA: *p≤0.05; n=6 per group.

FIG. 5. (A) Electrochemical impedance spectroscopy (EIS) curves for WE43, Mg, unprocessed MMNC, UST MMNC, and UST Rolled MMNC. The inset shows the simulated equivalent circuit used to analyze the Nyquist plots. (B) Corrosion resistance (Rp) of the samples, with UST Rolled MMNCs exhibiting a significant increase compared to the other samples. One-way ANOVA: **p≤0.01, ***p≤0.001; n=6 per group.

FIG. 6. (A-D) Corrosion rate (in mm per year) calculated from mass loss percentage according to ASTM G31 for 1-day (A), 3-day (B), 7-day (C), and 10-day (D) immersion tests. (E-1) Corrosion rates within each sample group (n=3), illustrating changes in corrosion behavior over time. UST Rolled MMNCs (1) demonstrated consistently stable corrosion rates, which were higher than the control WE43 (E) and Mg (F), and generally higher than UST MMNCs (H). Unprocessed MMNCs (G) exhibited a rapid increase in corrosion rate with immersion time, while UST MMNCs showed a similar trend but at one-fifth of the magnitude. In contrast, UST Rolled MMNCs maintained a more stable corrosion rate throughout the tests. One-way ANOVA: *p<0.05, ***p≤0.001, ****p≤0.0001; n=3 per group.

### DETAILED DESCRIPTION

Embodiments of the invention are based on the fabrication of a novel MMNC that involves incorporating Sc and Sr as alloying elements and BG as nano-reinforcements using ultrasonic melt treatment, followed by a hot rolling process. The effects of ultrasonic melt processing and rolling on the microstructure, as well as the mechanical properties and corrosion resistance, of the MMNC was evaluated. The results were compared with those of pure magnesium, unprocessed MMNCs, and WE43, a commercial biocompatible magnesium-based alloy, as a control. The fabrication of the MMNC composition using melt ultrasound processing and hot rolling resulted in significant increases in the yield stress and ultimate compressive strength of samples, while decreasing the elongation at break. Likewise, the sonication independently increased the corrosion resistance of the samples when immersed in a static HBSS solution and improved the corrosion behavior, as seen in the electrochemical analysis provided herein. The sonication of the melt did not significantly change the mechanical properties of the MMNC but did result in lower corrosion rates. Further processing of samples with hot rolling increased the corrosion rate, but it remained lower than the unprocessed and UST-processed MMNCs.

### Definitions

In this specification, if any numerical ranges are provided, the ranges include also the upper and lower values. The open term “comprise” also includes a closed term “consisting of” as one option. The dimensions disclosed herein can be detected microscopically, such as by using optical microscopy, for example from microscopy images. The images may be analyzed using a dedicated software, which may be provided by the microscopy manufacturer, such as to determine the dimensions and/or size distribution from an image.

Unless defined otherwise, all technical and scientific terms used herein have the same meaning as commonly understood by one of ordinary skill in the art to which the invention belongs.

In this specification, except where the context requires otherwise, the words “comprise”, “comprises” and “comprising” means “include”, “includes” and “including”, respectively. That is, when the invention is described or defined as comprising specified features, various embodiments of the same invention may also include additional features.

The percentages presented herein are weight percentages, unless otherwise indicated. The phrases “weight percent”, “percent by weight”, “by weight”, “% (w/w)” and “wt %” may be used interchangeably and are intended to denote the percent by weight based on the total expressed composition, unless otherwise explicitly stated.

The term “biodegradable” refers to material which, when in contact with biological environment, will degrade and/or decompose, either fully or partially. The degradation may be caused or accelerated by the biological environment, and it may be controlled by selecting suitable composition and properties of the material. The biological environment may refer to a body, such as one or more parts and/or tissues of body which are implantable with the present implants, for example bone, cartilage and/or other associated tissues or other body parts. The materials may be fully biodegradable in a body.

The term “bioresorbable” refers to material, such as metals or alloys thereof, polymers, glass and/or ceramics, which in contact with biological environment such biological tissues and/or physiological fluids will, following placement, degrade, resorb and/or absorb into the environment, either fully or partially, while maintaining its mechanical properties for a certain period of time. More particularly “bioresorbable”, for example in context with metals or alloys thereof, glass and/or ceramics, may refer to such materials which degrade safely within the body. The materials may be fully bioresorbable in a body.

The terms “bioresorbable”, “biodegradable”, “biosoluble”, “bioabsorbable”, biocorridible and “bioerodible”, with or without prefix “bio”, are often used interchangeably, and the terms may at least overlap. If applicable, the terms may be used interchangeably also herein. However the term “biodegradable” is intended to cover all the other terms as it does not specify the type of degradation or what will happen to the degradation products in the biological environment. The “biodegradable” may refer to biodegradable in body and/or biodegradable in contact with (biological) tissue and/or body tissue.

The term decomposability may be defined here as (biology) to break down (organic matter) or (of organic matter) to be broken down physically and chemically; (chemistry) to break down or cause to break down into simpler chemical compounds or to break up or separate into constituent parts. Degradability may refer to the state or quality of being susceptible to breakdown or decomposition.

Herein and hereafter “optional” or “optionally” denotes that the subsequently described event or circumstance may but need not occur, and that the description includes instances where the event or circumstance occurs and instances in which it does not. “Comprises” or “comprising” denotes that the subsequently described set may, but need not, include other elements.

### Detailed Description of Certain Embodiments

Accordingly, one embodiment pertains to a method of producing MMNC that involves heating a combination of magnesium, scandium, bioglass (BG) and strontium at a temperature and time to melt one or more constituents of the combination to form a melted mixture; sonicating the melted mixture to produce an ultrasonic processed melt; cooling the ultrasonic processed melt in a container to form a MMNC matrix; and hot-rolling the MMNC matrix. In a specific embodiment, the MMNC matrix is heated at a temperature and time, optionally 400° C. for 6 h, to homogenize the microstructure of the matrix and placed in second container. The hot-rolling step is conducted at a temperature, optionally 400° C., and rolled at a pressure to reduce grain size of the MMNC matrix. In a more specific embodiment, the MMNC matrix is formed into cylinders and hot-rolled to 4-7 mm diameter. According to other embodiments, the BG is diopside BG.

The present application provides a medical device, such as an implant or part thereof, comprising MMNC. The MMNC in the medical device may be present as such and/or it may be present as composite material together with one or more further material(s).

The implant may be any suitable medical device which is designed, arranged and/or intended to be inserted into living tissue, either fully or partially, such as into a body, for example into a bone. The implant may be used for bone-to-bone, soft tissue-to-bone, soft tissue-into-bone and/or soft tissue-to-soft tissue fixation. The implant is biocompatible with living tissue. The implant, or at least the magnesium alloy therein, may be biodegradable, such as fully biodegradable. The implant may be or may be provided in the form of an orthopedic implant, for example a bone implant. The implant may or may not contain other parts in addition to a part comprising or consisting of the magnesium alloy disclosed herein. The optional other part(s) may or may not be biodegradable. However fully biodegradable implants are preferred for the present uses. The properties of the present MMNCs and object formed from the MMNCs are especially suitable for orthopedic implants, which benefit the enhanced mechanical and chemical properties of the material, such as bending and shear strength, elasticity, controlled and predictable biodegradability, and other properties disclosed herein.

Other methods relate to a method of repairing a surgical bone site comprising placing an MMNC implant described herein at the surgical bone site.

A fundamental characteristic measure for microstructures of metallic materials is the average grain size, which may be determined for example by using linear intercept method, such as ASTM E1112-24 linear intercept method. A suitable software may be used to analyze microscopic images and to calculate and provide the average grain size.

### EXAMPLES

### 1. Materials and Methods

### 1.1. Synthesis of Diopside BG

Diopside (CaMgSi₂O₆) powder was synthesized using a sol-gel method: 2.1 g of calcium nitrate tetrahydrate (Acros Organics, 99+%, Geel, Belgium) and 2.0 g of magnesium chloride hexahydrate (Acros Organics, 99%) were dissolved in 200-proof ethanol (Decon Labs, King of Prussia, PA, USA). The solution was magnetically stirred at 250 rpm for 30 min at room temperature. Then, 20 mL of tetraethyl orthosilicate (Acros Organics, 98%) was added to the solution and stirred at 450 rpm for 24 h at 80° C. until a gel was formed. The gel was then dried at 100° C. for 5 days to obtain a xerogel, which was then manually milled with a mortar and pestle until an evenly fine powder was reached. The powder was calcinated in a furnace at 850° C. for 2 h.

### 1.2. Casting of MMNC

A negative sand mold was made using an oil-based sand (PMC Supplies LLC, Lake Katrine, NY, USA) with a riser and sprue to reach approximate laminar flow into the cavity. For the melting, magnesium (ThermoScientific, Waltham, MA, USA, 99.8% pure), scandium (Luciteria Science, Olympia, WA, USA 99.95% pure, 3 wt %), and the BG (0.5 wt %) were added to a graphite crucible and heated to 930° C. (1700° F.) under a constant argon atmosphere. The strontium (Luciteria Science, 99.9% pure, 0.3 wt %) was added last to reduce its oxidation. A graphite rod was inserted into the melt and stirred for 30 s to begin mixing. As a control, magnesium was cast using 99.8% pure Mg. WE43 (Goodfellow, Huntingdon, UK, Mg93-Y4-Nd3) was sourced as a commercialized control.

### 1.3. Ultrasonic Melt Processing

Ultrasound processing of the melt was conducted using a Hielscher UP200st (Hielscher Ultrasonics, Teltow, Germany), equipped with a ceramic sonotrode and an air circulation system to keep the transducer cool during the process. Prior to insertion, the temperature of the furnace was increased to 980° C. (1800° F.), and the sonotrode was pre-heated above the open furnace to prevent significant melt solidification on the probe. The probe was inserted into the melt approximately one centimeter from the bottom of the crucible. The melt underwent a 1 min treatment consisting of 6 cycles of 6 s on and 4 s off at a power output of 1 watt at 50% amplitude (maximum amplitude 35 μm). Shortly after the treatment was completed, slag was removed, and the entire melt was cast into a sand mold and allowed to cool for 2 h before extraction. The riser and sprue were cut and excluded from further analysis due to their non-uniform microstructure and inclusions.

### 1.4. Heat Treatment

Heat treatment was performed at 400° C. for 6 h to homogenize the microstructure. Samples were placed in quartz tubes topped with aluminum foil to prevent surface oxidation. After heating, samples were cooled to room temperature under ambient conditions while remaining in the quartz tubes.

### 1.5. Hot Rolling

Samples were machined to 6.3 mm diameter and pre-heated for one hour at 400° C. in aluminum foil-topped quartz tubes. Samples were removed from the tubes and rolled to 6.0 mm diameter. A second pre-heating and rolling to 5.0 mm diameter was performed to further reduce grain sizes.

### 1.6. Microstructural Characterization

Samples were cut using a wafering saw (Dimost, Suzhou, China, CT-2300), cleaned with 70% ethanol, and polished using an EXAKT 400 CS Microgrinder (Exakt Technologies, Inc., Oklahoma City, OK, USA) with ascending grits, finishing with 1200 grit. Polishing was performed with a clean felt pad and 0.5 μm alumina particles. After polishing, samples were cleaned with 70% ethanol and rinsed, then sonicated for 3 min in acetone to remove any surface contamination or organic materials. Samples were then etched in a nitric acid, acetic acid, and polyethylene glycol solution for approximately thirty seconds before rinsing with ethanol and deionized water to stop the etching process. A Keyence VHX microscope was used for microstructural imaging at 200× and 700× magnification.

After etching, images were analyzed for grain size measurements using ASTM E112 standards [33]. Mean lineal intercepts were calculated using ImageJ (version 153k, ImageJ, Bethesda, MD, USA) for measurements. In total, 11 replicates were randomly selected from various metallography images, and calculated grain sizes were recorded.

Further microstructural analysis was performed using scanning electron microscopy (SEM; Zeiss, Oberkochen, Germany, ULTRA-55 FEG) equipped with an energy-dispersive spectrometer (EDS; Noran System 7). The accelerating voltage and aperture size were 20 kV and 30 μm. Samples were coated with gold (Au) using a sputter coater (Quorum, Lewes, UK, EMS150T ES) to avoid surface charge on the samples. Furthermore, the morphology and size of the BG nanoparticles were observed using transmission electron microscopy (TEM, JEM-1011; JEOL, Tokyo, Japan) to examine the morphology and size of the synthesized BG nanoparticles after calcination.

### 1.7. Compression Testing

Compression testing was conducted in accordance with ASTM E9 standards [34]. Samples were cut to a length-to-diameter ratio of 2:1, assuring that the two faces were as parallel as possible. Tests were conducted on a TestResources model 313 with a crosshead speed of 0.5 mm/mm/min. Yield stress (σY) was determined by identifying the linear elastic region of the compression test curve and applying a 0.2% offset. Ultimate compressive strength (UCS) was calculated as the maximum stress recorded during the compression test. The maximum compressive strain at the point of fracture was measured and reported as the elongation at break (% elongation).

### 1.8. Electrochemical Corrosion Testing

Samples were subject to electrochemical analysis using a potentiostat (Biologic, Sp-50e, Biologic, Seyssinet-Pariset, France). Samples were cut to 3 mm segments from the rods and sequentially polished with 320-, 600-, and 1200-grit silicon carbide paper. Samples for corrosion testing were cut from different regions to evaluate the homogeneity of the microstructure and composition across the rods using corrosion data. WE43, Mg, ultrasonically treated (UST) MMNC, and unprocessed MMNC were loaded into a conductive sample holder with 0.5 cm² of surface area exposed for corrosion testing. However, the 5.3 mm diameter of the UST Rolled MMNC sample could not be fit into the commercial sample holder without causing leakage. As a result, these samples were wrapped with conductive wire and mounted in resin before polishing. The exposed surface area for the UST Rolled MMNC composition was 0.19 cm².

Samples were submerged in 200 mL of Hanks Balanced Salt Solution (HBSS) with Ca and Mg (Cytiva) adjusted to pH 7.4±0.05. The solution temperature was maintained at 37±1° C. via water bath. Corrosion properties were investigated using a traditional three-electrode electrochemical cell. Each experimental condition involved the working electrode connected to a graphite counter electrode and an Ag/AgCl reference electrode attached via a bridge tube.

Electrochemical impedance spectroscopy (EIS) and potentiodynamic polarization tests (PDP) were captured and analyzed using the EC-Lab®v11.43 (Biologic, France) software. The test began with a 55 min open circuit voltage (OCV) test. Single sine EIS analysis immediately followed OCV at frequency ranging from 100,000 Hz to 0.01 Hz and amplitude of 10 mV. EIS data were reported as a Nyquist plot and analyzed using EC-Labs software v11.43 (Biologic, France) with the equivalent circuit. PDP was conducted at a scanning rate of 10 mV/min and from −0.2 V to 0.2 V of OCV immediately after the EIS test. Tafel fit lines derived from slopes at ±20 mV and ±50 mV were used to extrapolate the corrosion current density (I_{corr}), and corrosion potential (E_{corr}) in the EC-Labs software v11.43. The corrosion rate was calculated with Equation (1) established in ASTM G59 [35]:

$$Corrosion Rate ( mmyr ) = 3.27 \times {10}^{- 3} \frac{{I}_{corr} EW}{ρ} \qquad ( 1 )$$

where I_{corr} is the corrosion current density (μA/cm²) of the sample, EW is the sample equivalent weight of 12.15 g/eq for Mg based on oxidation from Mg to Mg²⁺, and p is the density of Mg considered to be 1.74 g/cm³. Six samples were analyzed in each condition.

### 1.9. Immersion Testing

Immersion testing was conducted in HBSS to measure corrosion rate. Samples were machined and cut to approximate diameters of 5 mm and lengths of 10 mm. After machining and cutting, samples were polished with 1200-grit sandpaper and washed in a sonicated acetone bath for 3 min to remove any oils or organic residues on the surface. Following ASTM G31 guidelines, the samples were weighed to an accuracy of ±0.001 g and immersed in pH-corrected HBSS (7.4±0.05) at a volume-to-surface area ratio of 0.2 mL/mm² [36]. Samples were immersed for 1, 3, 7, or 10 days in HBSS in glass beakers covered with Parafilm. Beakers were immersed in a water bath at 37° C. to mimic body temperatures and HBSS was refreshed every 72 h.

Corroded samples were removed from HBSS at the end of incubation and dried in an oven at 100° C. to stop further corrosion. Corroded weights were measured using a LEADZM electronic balance and recorded to an accuracy of ±0.001 g. Corrosion products were removed using 200 g/L chromic acid immersion for 10 min followed by a 5 min rinse in 200-proof ethanol. Samples were weighed after washing, and the change in mass was used to calculate the corrosion rate via mass loss percent following Equation (2), where Δm is the change in mass calculated after corrosion products were removed. A is the surface area of the samples, T is the time immersed in hours, and D is the density of the samples.

$$Corrosion Rate ( mmyr ) = ( 87 , 600 \times Δ m ) / ( A \times T \times D ) \qquad ( 2 )$$

### 1.10 Statistical Analysis

Metallographic imaging was performed on 1-2 samples per group, selected from different sections of the cast rods, and representative images were chosen. Mechanical testing samples were cut from randomized sections of the cast rods, with 5 replicates per group. Electrochemical testing was conducted with 6 replicates per group. For immersion testing, 3 replicates per group were used for each time point. The results are presented as mean±standard deviation. Statistical analyses were carried out using one-way ANOVA with Tukey's post hoc analysis in GraphPad Prism 10.2.3 software (San Diego, CA, USA). Statistical significance was considered at p s 0.05, with results reported as follows: *p≤0.05, **p≤0.01, ***p≤0.001, ****p≤0.0001.

### 2. Results

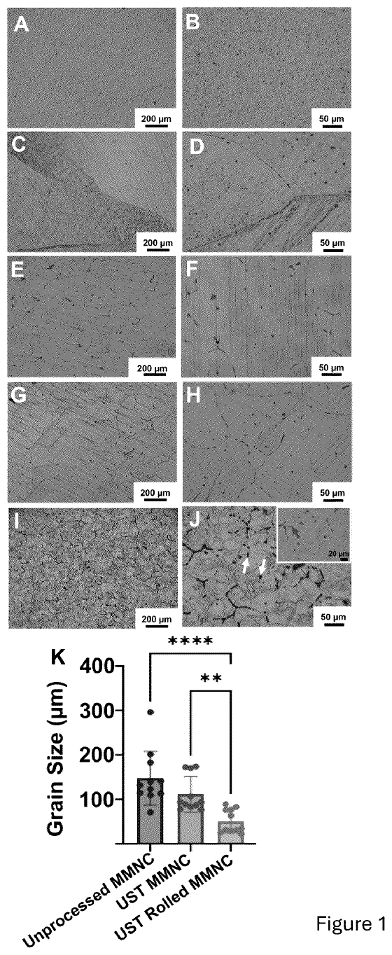

FIG. 1 shows the polished and etched surfaces of each material. In UST and UST Rolled MMNCs, the increased concentration of dark regions along the grain boundaries and within the grains likely corresponds to diopside bioactive glass-ceramic (BG) nanoparticles. Rolling led to a significant reduction in grain size and induced deformation twinning within the MMNC matrix. The rolled samples exhibit increased segregation of BG along the grain boundaries, as the grain deformation during hot rolling drives the nanoparticles toward these regions. Compared to Mg, UST MMNCs showed a more refined grain microstructure, with BG nano-reinforcements dispersed throughout the Mg matrix. However, when rolling is performed on UST MMNCs, the grain size is reduced. Moreover, the simultaneous application of high temperature and pressure enhanced the nucleation of intermetallics, primarily along the grain boundaries and at the sites of nano-reinforcements. Overall, both UST MMNCs and UST Rolled MMNCs displayed a significantly more refined grain structure, with smaller and more uniform grain sizes compared to Mg. UST MMNCs and unprocessed MMNCs showed similar grain sizes, with UST MMNC having smaller grains (112.2±40.2, 148.4 60.75, respectively, p>0.05). UST Rolled MMNCs showed a grain size of 51.78±26.56, significantly lower than unprocessed MMNCs (p<0.0001) and UST Rolled MMNCs (p<0.01).

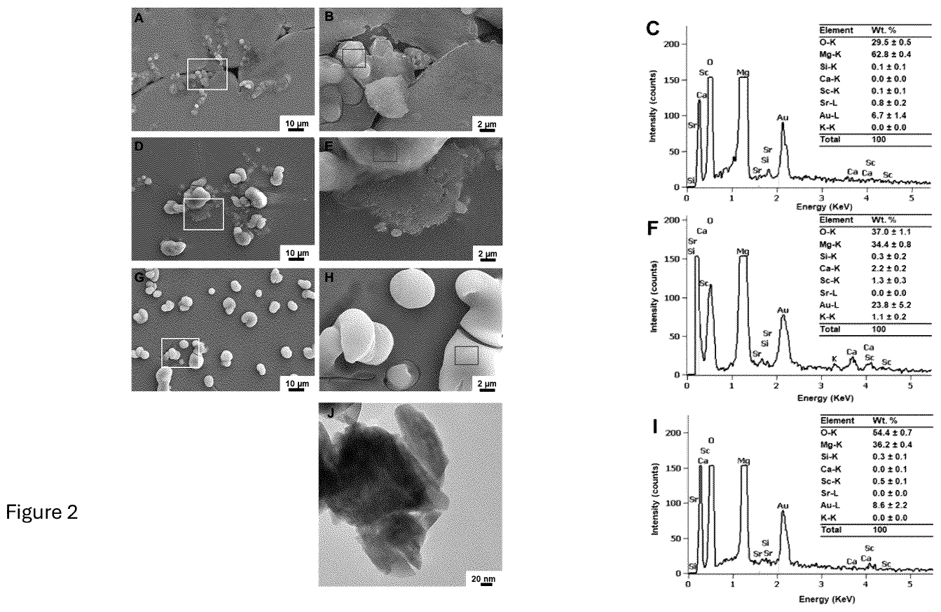

FIG. 2 presents the SEM images of unprocessed MMNC, UST MMNC, and UST Rolled MMNC and the results of EDS analysis. In FIG. 2A, precipitates are observed accumulating along the grain boundaries, which exhibit a discontinuous interface, indicating poor matrix integrity. The inset in FIG. 2A is enlarged in FIG. 2B, which highlights the EDS analysis area predominantly composed of Mg, O, and Sr (FIG. 2C). For UST MMNC, the dispersion of particles within the matrix appears more homogeneous, demonstrating improved integrity compared to the unprocessed sample (FIG. 2D,E). The selected inset in FIG. 2E reveals a significant presence of Si (0.3±0.2 wt. %) and Ca (2.2±0.2 wt. %), indicating the dispersion of BG nanoparticle aggregates. However, some BG nanoparticles remain aggregated and exhibit irregular shapes after the UST process. Conversely, UST Rolled MMNC displays smaller, round particles with a more uniform distribution (FIG. 2G,H). The EDS results for UST Rolled MMNC (FIG. 21) indicate the presence of Si (0.3±0.1 wt. %), indicating the incorporation of BG. In addition, the Sc concentration was highest in the following order: UST MMNC (1.3±0.3 wt. %), UST Rolled MMNC (0.5±0.1 wt. %), and unprocessed MMNC (0.1±0.1 wt. %). FIG. 2J demonstrates the TEM image of BG nanoparticles after calcination at 850° C. for 2 h. It is composed of 3-5 nanoparticles with varying morphologies, ranging from elongated to semi-spherical shapes.

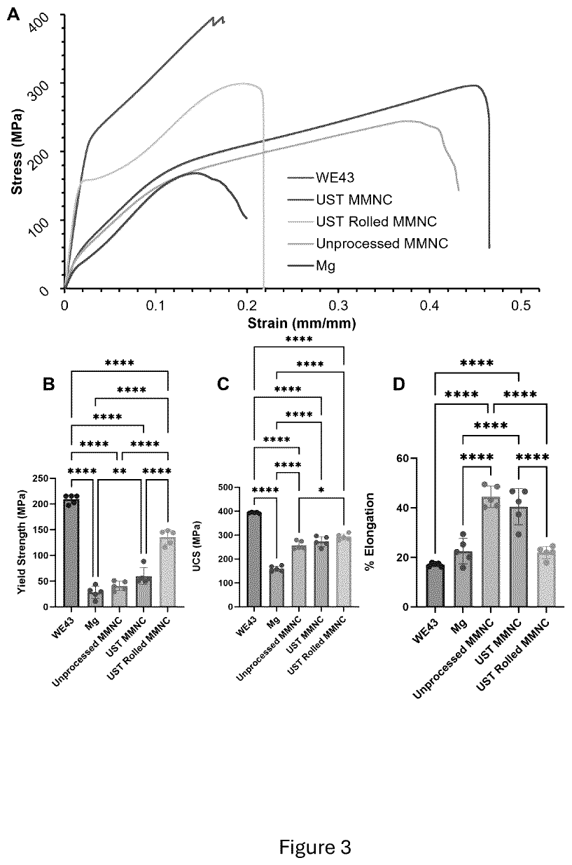

Compression testing was performed on the samples to evaluate their potential as load-bearing bone implants. WE43 was used as a control due to its well-documented properties and its established commercial use in bone implants. WE43 has been reported to have a yield stress, cy, of about 240 MPa, an ultimate compressive stress, UCS, of approximately 400 MPa, and a percent elongation at break of about 17% [37]. These reported values are comparable to our WE43 measurements of 6y (209±8.71), UCS (393±2.8), and percent elongation (17.02±0.74) (FIG. 3 and Table 1). Compression testing showed that the Mg had a significantly lower (p<0.05) yield stress than the UST MMNCs and UST Rolled MMNCs, but not compared to unprocessed MMNC. The hot rolling process led to a significantly higher UCS compared to the UST MMNCs, while having no significant effect on the yield strength. Similarly, the percent elongation at break for rolled MMNCs was significantly lower (p<0.05), showing a reduction of 45% compared to the unrolled UST samples.

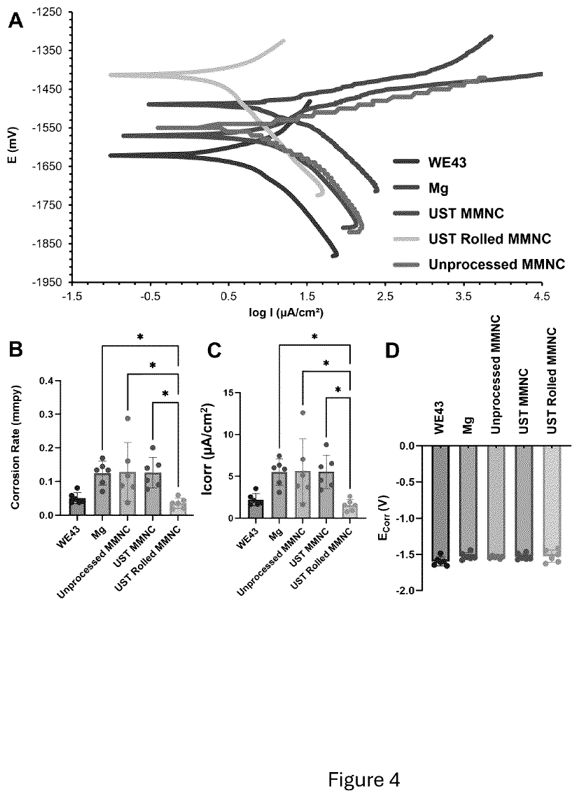

FIG. 4A displays representative PDP curves selected based on proximity to the average corrosion rate of six replicates. FIG. 4B-D display the average corrosion rates, corrosion current densities (I_{corr}), and corrosion potentials (E_{corr}) of the experimental groups. Commercially developed WE43 and Mg were analyzed as control groups to compare the findings of the experimental MMNCs subjected to UST and rolling processes.

Corrosion current density indicates the rate of corrosion, while corrosion potential represents the equilibrium between oxidation and reduction reactions at the metal's surface [38]. Samples with higher, or more positive, corrosion potentials are more likely to gain electrons rather than lose them, reducing their tendency to corrode [38]. Furthermore, a lower corrosion current density suggests a lower corrosion rate [38]. Both the unprocessed MMNC and UST MMNC were characterized by corrosion potentials near that of Mg and higher than WE43. The UST Rolled MMNCs displayed the highest E_{corr} of −1.44±0.08 V, compared to other groups, including WE43 which displayed an E_{corr} of −1.6±0.06 V.

Tafel-derived corrosion current densities show that there are significant differences (p<0.05) between I_{corr} of the UST Rolled MMNC and that of the UST MMNC. The I_{corr} of the UST Rolled MMNC was 1.55±0.67 μA/cm² compared to the I_{corr} of 5.53±2.01 μA/cm² in the UST MMNC. Significant differences also exist between the UST Rolled MMNC and Mg (I_{corr}=5.48±1.58 μA/cm²) and the unprocessed MMNC, where I_{corr} was 5.63±3.85 μA/cm². The near 110 mV difference in E_{corr}, along with these findings, suggests that the changes induced by the rolling process are significant, leading to increased corrosion resistance in the UST Rolled MMNC. These modifications result in the UST Rolled MMNC exhibiting a current density similar to that of commercialized WE43, although the difference between the two groups is not statistically significant (p>0.05).

The UST Rolled MMNC displayed the lowest corrosion rate of all groups. The corrosion rate of the UST Rolled MMNC was 0.03±0.02-a 4-fold reduction in corrosion rate compared to rates of 0.13 mm per year (mmpy) in the UST MMNC, Mg, and unprocessed MMNC. No significant difference (p>0.05) was determined between corrosion rates of UST Rolled MMNC and WE43. The trend in corrosion rates corresponds directly to the trend in current density and corrosion potential. Furthermore, the trends are justified by the interrelation of corrosion rate and current density through the formula established in ASTM G59 (Equation (1)) [35]. It is also notable that the unprocessed MMNC exhibited greater variation in corrosion, likely due to non-uniform microstructures across samples. This underscores the role of UST in producing more uniform microstructures and consistent corrosion rates.

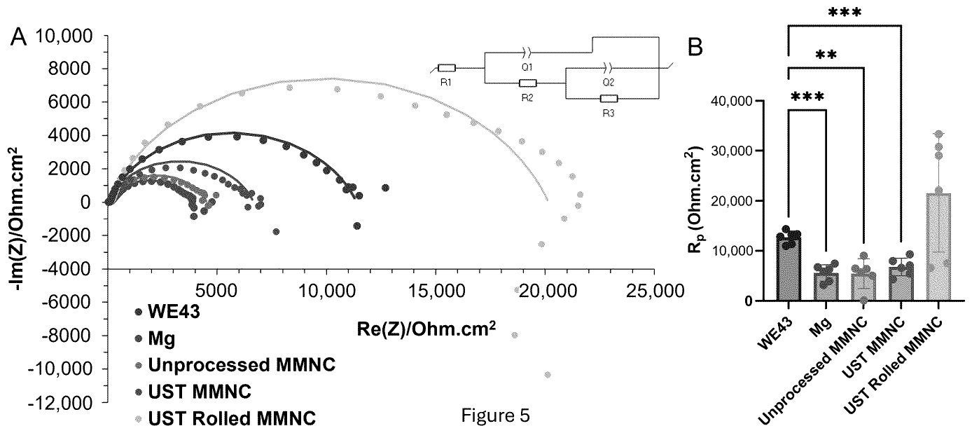

FIG. 5A,B depict the results of electrochemical impedance spectroscopy (EIS) for the representative curves closest to the average for each condition. The simulated equivalent circuit used to evaluate each Nyquist plot can be found inlayed in FIG. 5A. This equivalent circuit was drafted based on the presence of both high- and low-frequency capacitive loops in the Nyquist plots of all samples [39]. Mg alloys characterized by this type of impedance behavior exhibit two porous layers, the outer layer of which can indicate the formation of a protective corrosion layer [39,40].

The layers are modeled using two elements: electrical resistance (R) and a capacitance/constant phase element (Q), with an impedance defined as Z_{Q}=Q₀(i.ω)ⁿ (Q₀=constant; i=imaginary number; ω=angular frequency; and n=coefficient between 0.5 and 1). When n approaches 1, it indicates near-ideal capacitance, suggesting an effective barrier to solution penetration [41]. The accuracy of the fitting models was evaluated by comparing the reduced chi-square (χ²/|Z|), which represents the ratio of observed experimental variance to theoretical variance. Although χ²/|Z| is less than 1, indicating minimal noise in the data, the model effectively fits the raw Nyquist data.

Among the values represented by the equivalent circuit, R₁ indicates the solution resistance (Rₛ) characteristic of the electrolytes in HBSS. R₂ and Q, indicate the resistance and capacitance, respectively, between the protective outer porous layer and solution. The charge transfer resistance and capacitance at the inner layer of the alloy are represented by R₃ and Q₂, respectively. EIS data extrapolated from equivalent circuits using EC-Lab software v11.43 are summarized in Table 2, with polarization resistance (Rp) being of particular significance. All six replicates of a single group were analyzed sequentially before proceeding to the next sample.

Q₁ for Mg (6.21±2.07 μF·cm⁻²·sⁿ), UST MMNC (6.88±4.02 μF·cm⁻²·sⁿ), and UST Rolled MMNC (6.25±6.34 μF·cm⁻²·sⁿ) shows smaller values compared to WE43 (8.38±3.55 μF·cm⁻²·sⁿ) and unprocessed MMNC (9.89±7.72 μF·cm⁻²·sⁿ), while the n₁ values are quite similar in all groups (0.68-0.71). On the other hand, Q₂ is significantly higher for UST Rolled MMNC (155.97±375.88 μF·cm⁻²·sⁿ) compared to the other groups and is a less ideal capacitance (n₂=0.75±0.33). The outer- and inner-layer resistance for UST Rolled MMNCs (R₂=1365.69±2545.17 Ohm·cm² and R₃=20,213.85±13,413.07 Ohm·cm²) show relatively higher values compared to the other groups (Table 2). Consequently, the UST Rolled MMNC displayed Rp of 21,579.54±11,839.93 Ohm·cm², almost two-fold higher than the next highest value of 13,006.51±1072.27 Ohm·cm² in the WE43 condition. These Rp values in Table 2 also correspond to the size of the capacitive loops in the Nyquist plot of each sample as larger capacitive loops correspond to higher resistance values and better protection against corrosion. The data in Table 2 show that UST rolling produced the most pronounced increase in the polarization resistance of the sample. Resistance at the outer porous layer of the samples was also highest in the UST Rolled MMNC group, at 1365.69±2545.17 Ohm·cm² compared to all other conditions within a range of 95.11-169.02 Ohm·cm².

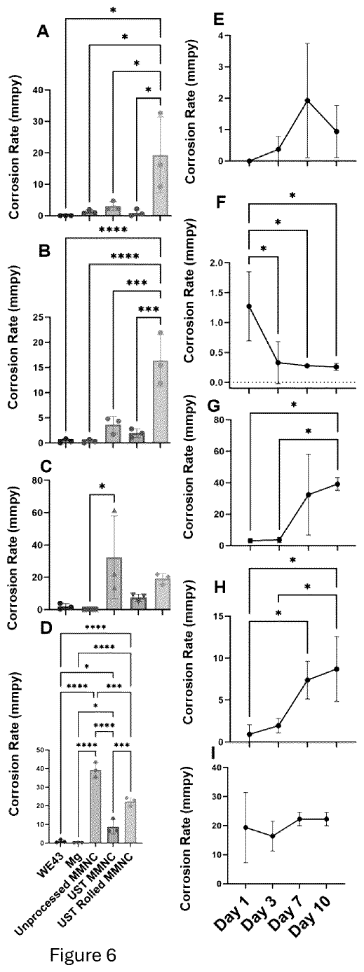

Although UST Rolled MMNC exhibited a significantly higher corrosion rate than all other samples at the initial immersion, its corrosion rate remained statistically constant over the incubation period. In contrast, other groups showed increasing corrosion rates with longer immersion times (FIG. 6, Table 3). After 7 days of immersion, unprocessed MMNC exhibited a significant increase in corrosion rate, rising ten-fold. Similarly, between days 3 and 7, UST MMNC showed a3.8-fold increase in corrosion rate and began showing statistically significant differences in corrosion rates compared to Mg.

### 3. Discussion

The above examples demonstrate the significant impact of ultrasonic melt treatment (UST) on the microstructure, mechanical properties, and corrosion resistance of a novel magnesium-based metal matrix nanocomposite (MMNC). We also investigated the effects of rolling on UST MMNC, finding improvements in the consistency of corrosion rates, an increase in yield stress, and a reduction in percent elongation at break. Additionally, regardless of the processing techniques, adding alloying elements and nano-reinforcements led to increased ultimate compressive strength and yield stress compared to Mg.

Ultrasonication of the melt was selected as the treatment method due to its ability to induce acoustic streaming and bubble cavitation in the melt [22,42,43]. Preliminary testing revealed inconsistent corrosion results with adding bioactive glass-ceramic nanoparticles (BG) at significantly higher weight percentages than the current 0.5 wt. %. Reducing the BG concentration minimized agglomeration, as the lower overall amount of BG reduced the tendency for clustering. Subsequently, ultrasonic melt treatment was used to de-agglomerate and disperse the BG uniformly within the melt while also degassing the melt through acoustic streaming and bubble cavitation effects. This uniformity in microstructure is evident in the metallography images shown in FIG. 1. However, the UST MMNC still exhibits large grains, potentially due to the low solidification rate of the melt in the sand mold. Previous research has shown that grain size significantly affects the corrosion rate of Mg [44,45]. Future work may involve optimizing the mold dimensions and mold material to enhance solidification rates. In addition, further refinement of the ultrasonic melt treatment process could help reduce grain sizes and achieve more controlled corrosion properties. Hot rolling was selected to reduce grain size and enhance the integration between the Mg matrix and BG reinforcements due to its simplicity and accessibility. However, this method is limited by the number of cycles achievable, as it necessitates a significant reduction in cross-sectional area during the process. Ultrasonication of the melt did not lead to a significant reduction in grain size. The primary goal of melt sonication is to de-agglomerate and disperse BG nanoparticles while inducing cavitation of trapped gas, thereby reducing structural defects and casting porosity. The enhanced dispersion of BG was expected to promote grain nucleation, potentially resulting in smaller grain sizes. However, due to the solidification rate of the melt in the sand mold and the subsequent heat treatment, the impact of ultrasonication on grain size was negligible. Emadi et al. demonstrated that ultrasonication of an AZ91E Mg alloy melt significantly influenced grain size after a 24-h heat treatment [46]. In contrast, metallographic imaging of AZ91E in their study revealed a considerable presence of secondary beta phase throughout the SEM images. At higher amplitudes, ultrasonication led to a reduction in the segregation of large beta-phase areas. In our case, however, the minimal presence of secondary phases in MMNCs due to the low levels of alloying elements resulted in a more negligible effect of ultrasonication on grain size.

The SEM/EDS results indicated that UST improved the wettability of the BG particles, leading to a more homogeneous dispersion compared to the unprocessed sample (FIG. 2A,D). The rolling process further enhanced this dispersion, as illustrated in FIG. 2G. The morphology of the BG particles underwent a significant transformation into a spherical shape, likely due to deformation and breakdown of BG nanoparticle aggregates during the rolling process. The shape of the reinforcing particles has been shown to influence the biodegradation of Mg/HA nanocomposites [47]. Similarly, in this study, the UST Rolled MMNC exhibited the lowest corrosion current density (1.55±0.67 μA/cm²), corrosion rate (0.03±0.02 mmpy), and polarization resistance (21,579.54±11,839.93 Ohm·cm²). However, the immersion results indicated an increased corrosion rate, necessitating a prolonged EIS test to elucidate the underlying reasons. One potential explanation could be the higher density of grain boundaries, which may have facilitated passivation during the initial stages of corrosion, as measured by electrochemical methods. Additionally, the presence of significant amounts of oxide could have contributed to the disintegration of the matrix, while solution penetration may have diminished corrosion resistance. Oxidation may have occurred during the heat treatment before rolling, as evidenced by SEM/EDS (FIG. 21). The UST Rolled MMNC had the highest O content of 54.4±0.7 wt. %. The homogeneous dispersion of the BG particles also increased yield strength, following the order UST Rolled MMNC>UST MMNC>unprocessed MMNC. In metal matrix composites, particle strengthening is a mechanism that enhances yield strength [48]. Furthermore, the shape, size, and quantity of the particles can significantly influence mechanical properties, such as crack initiation within the matrix. Smaller particles with a narrow size distribution tend to reduce the likelihood of crack formation.

Hot rolling also offers additional benefits, such as increased pressure and temperature, enhancing the nucleation and growth of intermetallic phases. This increases yield stress and ultimate compressive strength (UCS) in the UST Rolled MMNC while reducing elongation at break. However, the formation of intermetallic phases can increase the risk of micro-galvanic pitting corrosion due to variations in electrochemical corrosion potentials relative to the Mg matrix. The segregation of intermetallics at grain boundaries, as in FIG. 1, can lead to pitting corrosion along these boundaries. This can result in intergranular corrosion, pit-to-crack transition, and early material failure. Another major drawback of the hot rolling process is the introduction of stress-induced twinning and microcracks. The roughness from twinning at the micron scale increases the surface area, which can significantly enhance pitting corrosion [49]. Likewise, microcracks formed during the significant plastic deformation during hot rolling may act as initiation sites for further pitting corrosion.

The corrosion behavior observed during electrochemical corrosion testing can be attributed to the microstructural changes induced by the UST rolling process. Specifically, the increased compressive residual stress and dislocation density contribute to enhanced recrystallization and grain refinement [50,51], while also promoting the closure of matrix-reinforcement interfacial gaps. High dislocation levels and subtle defects introduced by the rolling process can create diffusion pathways for elements to reach the metal surface, forming a protective oxide layer that enhances corrosion resistance [51]. Corrosion rates calculated via PDP analysis are supported by a study from Deng et al., examining the corrosion behavior of Mg alloys. They concluded that hot rolling significantly reduced the corrosion rate by up to three times, with the effect being temperature-dependent [52].

Generally, the charge transfer resistance (R₃ in Table 2) indicates the dissolution rate of Mg, and higher values equate to slower rates of dissolution, specifically lower corrosion rates [53]. When analyzed, the charge transfer resistance values (R₃) were highest in the WE43 and UST Rolled MMNCs, at 12,873.20±1074.57 Ohm·cm² and 20,213.85±13,413.07 Ohm·cm², respectively. These data demonstrate over a two-fold difference from the UST MMNC, Mg, and unprocessed MMNCs compared to WE43 and a four-fold difference compared to the UST Rolled MMNCs. These conclusions further underscore the significant impact of integrated UST and rolling processes on the corrosion resistance of MMNC. It is also important to note that the electrochemical behavior of the MMNCs is expected to change as the surfaces of the samples are oxidized and corroded. Due to this expectation, further study into the electrochemical behavior of already-immersed samples should be conducted to validate existing EIS and immersion discrepancies, as well as to study the possible beneficial effects of creating a passivation layer via immersion prior to electrochemical studies.

Immersion testing was used to confirm the corrosion rate of MMNC calculated from electrochemical testing. However, extended immersion testing, compared to electrochemical testing, may accelerate pit formation and the transition from pits to cracks due to micro-galvanic corrosion between the matrix, intermetallics, and nano-reinforcements, potentially leading to significantly higher corrosion rates [54]. The corrosion of the Mg matrix is evident from the formation of small bubbles on the sample surfaces as immersion time increases. While similar bubbles are observed on electrochemical corrosion samples, they appear at a much smaller scale and in fewer quantities. Variation may also be due to the creation of a passivation layer during the initial stages of the electrochemical corrosion test, prior to the EIS and PDP measurements.

The study of Mg-based alloys and composites is well established, with extensive research exploring a variety of testing media for these samples. Initially, solutions of NaCl had been used according to ASTM standards, but due to the significant differences in ionic compositions, physiological comparisons between these results and in vivo studies showed significant differences in the corrosion behavior and resulting corrosion products [55]. Due to these differences, immersion tests have evolved to include specific valid compositions such as Hanks Balanced Salt Solutions (HBSS), simulated body fluid (SBF), and Dulbecco's Phosphate-Buffered Saline (DPBS). Each of these fluids have various ionic profiles, specially formulated to imitate some aspect of physiological solutions.

Mena-Morcillo and Veleva recently studied the effect that varying the immersion media had on the corrosion rate of AZ31 and AZ91 [56]. They observed a significant variation in corrosion rates between HBSS, SBF, and Ringer's solution. Specifically, HBSS resulted in a mass loss rate approximately three times lower than that observed in SBF. Additionally, Mei et al. discussed criteria for selecting the appropriate immersion media in their 2020 review [55]. They concluded that simulated body fluids like SBF and HBSS were suitable for open immersion tests due to the lack of sugars and other elements that would cause microbial contaminations to tamper with the data. They also concluded that the corrosion rate of Mg samples in HBSS-like media would be “revelated to a lesser extent” when compared to NaCl solutions due to the various ionic interactions between the Mg matrix and the ionic solution. However, the human body has a complex ionically stable and pH-stable condition that significantly contributes to the behavior of the implants in situ.

It is widely reported that static immersion tests, like the one we conducted, often show higher corrosion rates compared to in vivo studies [57,58,59]. This discrepancy arises because in vivo specimens experience a constantly changing environment due to the circulatory system, which maintains a homeostatic ion exchange. Additionally, the presence of enzymes and proteins in vivo can contribute to the formation of protective layers.

Although UST and integrated UST rolling processes are effective in the microstructural refinement of MMNC, further optimization can be achieved in future regarding the ultrasound intensity, duration, amplitude, and hot rolling temperature to further reduce grain sizes, de-agglomerate and disperse nano-reinforcements, and reduce intermetallic and twining formation. Further optimization of MMNC composition and processing may enhance microstructure, corrosion resistance, and mechanical properties in future studies. In future studies, the formation of corrosion products will be investigated to better understand the corrosion mechanisms and the effects of biomineralization on corrosion rates. Other severe plastic deformation methods can also be used to reduce grain sizes, thereby elucidating the effects of processing on microstructure and resulting corrosion and mechanical properties.

The fabrication of a novel MMNC composition using ultrasonic melt processing and hot rolling resulted in significant increases in the yield stress and ultimate compressive strength of samples while decreasing the elongation at break. Likewise, ultrasonication independently increased the corrosion resistance of the samples when immersed in a static HBSS solution and improved the corrosion behavior, as seen in the electrochemical analysis. The sonication of the melt did not significantly change the mechanical properties of the MMNC but did result in lower corrosion rates. Further processing of samples with hot rolling increased the corrosion rate, but it remained lower than the unprocessed and UST-processed MMNCs.

### REFERENCES

1. Paiva, J. C. C.; Oliveira, L.; Vaz, M. F.; Costa-De-Oliveira, S. Biodegradable Bone Implants as a New Hope to Reduce Device-Associated Infections—A Systematic Review. Bioengineering 2022, 9, 409.
2. Prasad, A. Bioabsorbable polymeric materials for biofilms and other biomedical applications: Recent and future trends. Mater. Today Proc. 2021, 44, 2447-2453. [[https://doi.org/10.1016/j.matpr.2020.12.489]
3. Hayes, J.S.; Richards, R. The use of titanium and stainless steel in fracture fixation. Expert Rev Med. Devices 2010, 7, 843-853.
4. Disegi, J.; Eschbach, L. Stainless steel in bone surgery. Injury 2000, 31, D2-D6.
5. Moghaddam, N. S.; Jahadakbar, A.; Amerinatanzi, A.; Skoracki, R.; Miller, M.; Dean, D.; Elahinia, M. Fixation release and the bone bandaid: A new bone fixation device paradigm. Bioengineering 2017, 4, 5.
6. Xiu, P.; Jia, Z.; Lv, J.; Yin, C.; Cheng, Y; Zhang, K.; Song, C.; Leng, H.; Zheng, Y.; Cai, H.; et al. Tailored surface treatment of 3D printed porous Ti6A14V by microarc oxidation for enhanced osseointegration via optimized bone in-growth patterns and interlocked bone/implant interface. ACS Appl. Mater. Interfaces 2016, 8, 17964-17975.
7. Moghaddam, N. S.; Andani, M.T.; Amerinatanzi, A.; Haberland, C.; Huff, S.; Miller, M.; Elahinia, M.; Dean, D. Metals for bone implants: Safety, design, and efficacy. Biomanuf. Rev 2016, 1, 1.
8. Savarino, L.; Maci, G.S.; Greco, M.; Baldini, N.; Giunti, A. Metal ion release from fracture fixation devices: A potential marker of implant failure. J. Biomed. Mater. Res. Part B Appl. Biomater. Off. J. Soc. Biomater. Jpn. Soc. Biomater. Aust. Soc. Biomater. Korean Soc. Biomater. 2008, 86, 389-395.
9. Blumenthal, N.C.; Posner, A.S.; Cosma, V.; Gross, U. The effect of glass-ceramic bone implant materials on the in vitro formation of hydroxyapatite. J. Biomed. Mater. Res. 1988, 22, 1033-1041.
10. Ibrahim, H.; Esfahani, S. N.; Poorganji, B.; Dean, D.; Elahinia, M. Resorbable bone fixation alloys, forming, and post-fabrication treatments. Mater. Sci. Eng. C 2017, 70, 870-888.
11. Reyes, C.D.; Petrie, T.A.; Burns, K. L.; Schwartz, Z.; Garcia, A.J. Biomolecular surface coating to enhance orthopaedic tissue healing and integration. Biomaterials 2007, 28, 3228-3235.
12. Andersson, T.; Agholme, F.; Aspenberg, P.; Tengvall, P. Surface immobilized zoledronate improves screw fixation in rat bone: A new method for the coating of metal implants. J. Mater. Sci. Mater. Med. 2010, 21, 3029-3037.
13. Savaedi, Z.; Mirzadeh, H.; Aghdam, R. M.; Mahmudi, R. Effect of grain size on the mechanical properties and bio-corrosion resistance of pure magnesium. J. Mater. Res. Technol. 2022, 19, 3100-3109.
14. Ma, N.; Peng, Q.; Li, X.; Li, H.; Zhang, J.; Tian, Y. Influence of scandium on corrosion properties and electrochemical behaviour of mg alloys in different media. Int. J. Electrochem. Sci. 2012, 7, 8020-8034.
15. Mushahary, D.; Sravanthi, R.; Li, Y.; Kumar, M.J.; Harishankar, N.; Hodgson, P.D.; Wen, C.; Pande, G. Zirconium, calcium, and strontium contents in magnesium based biodegradable alloys modulate the efficiency of implant-induced osseointegration. Int. J. Nanomed. 2013, 8, 2887-2902.
16. Thakur, B.; Barve, S.; Pesode, P. Investigation on mechanical properties of AZ31B magnesium alloy manufactured by stir casting process. J. Mech. Behav. Biomed. Mater. 2023, 138, 105641.
17. Sathishkumar, P.; Deepakaravind, V.; Gopal, P.; Azhagiri, P. Analysis the mechanical properties and material characterization on magnesium metal matrix nano composites through stir casting process. Mater. Today Proc. 2021, 46, 7436-7441.
18. Ponappa, K.; Aravindan, S.; Rao, P. Magnesium Metal-Matrix Composites-Types and Fabrication Approaches. In Advances in Corrosion Control of Magnesium and Its Alloys; CRC Press: Boca Raton, FL, USA, 2023; pp. 23-34.
19. Arora, G.S.; Saxena, K.K.; Mohammed, K.A.; Prakash, C.; Dixit, S. Manufacturing techniques for Mg-Based metal matrix composite with different reinforcements. Crystals 2022, 12, 945.
20. Yin, Z.; Le, Q.; Chen, X.; Jia, Y. The grain refinement of Mg alloy subjected to dual-frequency ultrasonic melt treatment: A physical and numerical simulation. J. Mater. Res. Technol. 2022, 21, 1554-1569.
21. Hu, W.; Le, Q.; Liao, Q.; Wang, T. Effects of Ultrasonic Treatment on Grain Refinement and Gas Removal in Magnesium Alloys. Crystals 2024, 14, 237.
22. Eskin, G. Cavitation mechanism of ultrasonic melt degassing. Ultrason. Sonochem. 1995, 2, S137-S141.
23. Lebon, G.B.; Tzanakis, I.; Pericleous, K.; Eskin, D.; Grant, P.S. Ultrasonic liquid metal processing: The essential role of cavitation bubbles in controlling acoustic streaming. Ultrason. Sonochem. 2019, 55, 243-255.
24. Fatemi-Varzaneh, S.; Zarei-Hanzaki, A.; Haghshenas, M. The room temperature mechanical properties of hot-rolled AZ31 magnesium alloy. J. Alloys Compd. 2009, 475, 126-130.
25. Cao, F.; Shi, Z.; Song, G.-L.; Liu, M.; Dargusch, M.S.; Atrens, A. Influence of hot rolling on the corrosion behavior of several Mg—X alloys. Corros. Sci. 2015, 90, 176-191.
26. He, F.; Lu, T.; Fang, X.; Li, Y.; Zuo, F.; Deng, X.; Ye, J. Effects of strontium amount on the mechanical strength and cell-biological performance of magnesium-strontium phosphate bioceramics for bone regeneration. Mater. Sci. Eng. C 2020, 112, 110892.
27. Bornapour, M.; Celikin, M.; Cerruti, M.; Pekguleryuz, M. Magnesium implant alloy with low levels of strontium and calcium: The third element effect and phase selection improve bio-corrosion resistance and mechanical performance. Mater. Sci. Eng. C 2014, 35, 267-282.
28. Bian, D.; Chu, X.; Xiao, J.; Tong, Z.; Huang, H.; Jia, Q.; Liu, J.; Li, W.; Yu, H.; He, Y.; et al. Design of single-phased magnesium alloys with typically high solubility rare earth elements for biomedical applications: Concept and proof. Bioact. Mater. 2023, 22, 180-200.
29. Liu, J.; Lin, Y.; Bian, D.; Wang, M.; Lin, Z.; Chu, X.; Li, W.; Liu, Y.; Shen, Z.; Liu, Y.; et al. In vitro and in vivo studies of Mg-30Sc alloys with different phase structure for potential usage within bone. Acta Biomater. 2019, 98, 50-66.
30. Sheweita, S.; Khoshhal, K. Calcium metabolism and oxidative stress in bone fractures: Role of antioxidants. Curr. Drug Metab. 2007, 8, 519-525.
31. Yoshizawa, S.; Brown, A.; Barchowsky, A.; Sfeir, C. Magnesium ion stimulation of bone marrow stromal cells enhances osteogenic activity, simulating the effect of magnesium alloy degradation. Acta Biomater. 2014, 10, 2834-2842.
32. Hing, K.A.; Revell, P.A.; Smith, N.; Buckland, T. Effect of silicon level on rate, quality and progression of bone healing within silicate-substituted porous hydroxyapatite scaffolds. Biomaterials 2006, 27, 5014-5026.
33. ASTM E112-24; Standard Test Methods for Detemining Average Grain Size. ASTM: West Conshohocken, PA, USA, 2024.
34. ASTM E9; Standard Test Methods of Compression Testing of Metallic Materials at Room Temperature. ASTM: West Conshohocken, PA, USA, 2019.
35. ASTM G59-97; Standard Test Method for Conducting Potentiodynamic Polarization Resistance Measurements. ASTM: West Conshohocken, PA, USA, 2023.
36. ASTM G31; Standard Guide for Laboratory Immersion Corrosion Testing of Metals. ASTM: West Conshohocken, PA, USA, 2021.
37. Lei, Y.; Zhan, M.; Xin, H.; Ma, L.; Yuan, Y.; Zhang, H.; Zheng, Z. Comparison of the Strain Rate Sensitivity in AZ31 and WE43 Magnesium Alloys under Different Loading Conditions. Crystals 2023, 13, 554.
38. Elias, A.C. Principles and Presentation of Corrosion; Jones, D.A., Ed.; Prentice Hall: Upper Saddle River, NJ, USA, 1996.
39. Feliu, S., Jr. Electrochemical Impedance Spectroscopy for the Measurement of the Corrosion Rate of Magnesium Alloys: Brief Review and Challenges. Metals 2020, 10, 775.
40. Kirkland, N.; Birbilis, N.; Staiger, M. Assessing the corrosion of biodegradable magnesium implants: A critical review of current methodologies and their limitations. Acta Biomater. 2012, 8, 925-936.
41. Bagha, P.S.; Paternoster, C.; Khakbiz, M.; Sheibani, S.; Gholami, N.; Mantovani, D. Surface Modification of an Absorbable Bimodal Fe—Mn—Ag Alloy by Nitrogen Plasma Immersion Ion Implantation. Materials 2023, 16, 1048.
42. Priyadarshi, A.; Khavari, M.; Subroto, T.; Prentice, P.; Pericleous, K.; Eskin, D.; Durodola, J.; Tzanakis, I. Mechanisms of ultrasonic de-agglomeration of oxides through in-situ high-speed observations and acoustic measurements. Ultrason. Sonochemistry 2021, 79, 105792.
43. Kudryashova, O.; Vorozhtsov, S.; Khrustalyov, A.; Stepkina, M. Ultrasonic dispersion of agglomerated particles in metal melt. In AIP Conference Proceedings; AIP Publishing: Melville, NY, USA, 2016.
44. Saha, P.; Roy, M.; Datta, M.K.; Lee, B.; Kumta, P.N. Effects of grain refinement on the biocorrosion and in vitro bioactivity of magnesium. Mater. Sci. Eng. C 2015, 57, 294-303.
45. Aung, N.N.; Zhou, W. Effect of grain size and twins on corrosion behaviour of AZ31 B magnesium alloy. Corros. Sci. 2010, 52, 589-594.
46. Emadi, P.; Andilab, B.; Ravindran, C. Effects of sonication amplitude on the microstructure and mechanical properties of AZ91E magnesium alloy. J. Magnes. Alloys 2022, 10, 3397-3405.
47. Razavi, M.; Huang, Y. Effect of hydroxyapatite (HA) nanoparticles shape on biodegradation of Mg/HA nanocomposites processed by high shear solidification/equal channel angular extrusion route. Mater. Lett. 2020, 267, 127541.
48. Yang, Z.; Fan, J.; Liu, Y.; Nie, J.; Yang, Z.; Kang, Y. Effect of the particle size and matrix strength on strengthening and damage process of the particle reinforced metal matrix composites. Materials 2021, 14, 675.
49. Gerashi, E.; Alizadeh, R.; Langdon, T.G. Effect of crystallographic texture and twinning on the corrosion behavior of Mg alloys: A review. J. Magnes. Alloys 2022, 10, 313-325.
50. Chen, H.; He, Z.; Lu, L. Correlation of surface features with corrosion behaviors of interstitial free steel processed by temper rolling. J. Mater. Sci. Technol. 2020, 36, 37-44.
51. Xu, H.; Tian, T.; Hua, B.; Zhan, W.; Niu, L.; Han, B.; Zhang, Q. Effect of in-situ rolling and heat treatment on microstructure, mechanical and corrosion properties of wire-arc additively manufactured 316L stainless steel. J. Mater. Res. Technol. 2023, 27, 3349-3361.
52. Deng, B.; Dai, Y.; Lin, J.; Zhang, D. Effect of rolling treatment on microstructure, mechanical properties, and corrosion properties of WE43 alloy. Materials 2022, 15, 3985.
53. Cao, C.-N.; Zhang, J.-Q. An Introduction to Electrochemical Impedance Spectroscopy; Science Press: Beijing, China, 2002.
54. Han, L.; Zhang, Z.; Dai, J.; Li, X.; Bai, J.; Huang, Z.; Guo, C.; Xue, F.; Chu, C. In vitro bio-corrosion behaviors of biodegradable AZ31B magnesium alloy under static stresses of different forms and magnitudes. J. Magnes. Alloys 2023, 11, 1043-1056.
55. Mei, D.; Lamaka, S.V.; Lu, X.; Zheludkevich, M. L. Selecting medium for corrosion testing of bioabsorbable magnesium and other metals—A critical review. Corros. Sci. 2020, 171, 108722.
56. Mena-Morcillo, E.; Veleva, L. Degradation of AZ31 and AZ91 magnesium alloys in different physiological media: Effect of surface layer stability on electrochemical behaviour. J. Magnes. Alloys 2020, 8, 667-675.
57. Sanchez, A. H. M.; Luthringer, B.J.; Feyerabend, F.; Willumeit, R. Mg and Mg alloys: How comparable are in vitro and in vivo corrosion rates?A review. Acta Biomater. 2015, 13, 16-31.
58. Myrissa, A.; Agha, N.A.; Lu, Y.; Martinelli, E.; Eichler, J.; Szakecs, G.; Kleinhans, C.; Willumeit-Römer, R.; Schafer, U.; Weinberg, A.-M. In vitro and in vivo comparison of binary Mg alloys and pure Mg. Mater. Sci. Eng. C 2016, 61, 865-874.
59. Walker, J.; Shadanbaz, S.; Kirkland, N.T.; Stace, E.; Woodfield, T.; Staiger, M.P.; Dias, G.J. Magnesium alloys: Predicting in vivo corrosion with in vitro immersion testing. J. Biomed. Mater. Res. Part B Appl. Biomater. 2012, 100B, 1134-1141.

What is claimed is:

1. A method of producing a biodegradable and biocompatible magnesium-based metal-matrix nanocomposite (MMNC), the method comprising;
heating a combination of magnesium, scandium, bioglass (BG) and strontium at a temperature and time to melt one or more constituents of the combination to form a melted mixture;
sonicating the melted mixture to produce an ultrasonic processed melt;
cooling the ultrasonic processed melt in a container to form a MMNC sample; and
hot-rolling the MMNC sample.

2. The method of claim 1, wherein the MMNC sample is heated at a temperature and time, optionally 400° C. for 6 h, to homogenize the microstructure of the matrix and placed in second container.

3. The method of claim 1, wherein the hot-rolling step is conducted at a temperature, optionally 400° C., and rolled at a pressure to reduce grain size of the MMNC matrix.

4. The method of claim 1, wherein the MMNC sample is formed into cylinders and hot-rolled to 4-7 mm diameter.

5. The method of, wherein the BG is diopside BG.

6. An implant comprised of the MMNC of claim 1.

7. A method of conducting surgery on a subject in need, the method comprising inserting in the subject in need an implant of claim 6.

8. The method of claim 7, wherein the implant is inserted into a bone.
